(19) United States
(12) Patent Application Publication
(10) Pub. No.: US 2023/0404806 A1
(43) Pub. Date: Dec. 21, 2023

(54) SYSTEM FOR CUTTING OCULAR TISSUE INTO ELEMENTARY PORTIONS

(71) Applicant: KERANOVA, Saint Etienne (FR)

(72) Inventors: Fabrizio ROMANO, Beynost (FR); Cyril MAUCLAIR, Challain La Potherie (FR); Emmanuel BAUBEAU, Saint Etienne (FR)

(21) Appl. No.: 18/251,197

(22) PCT Filed: Oct. 28, 2021
(86) PCT No.: PCT/EP2021/080018

§ 371 Date: Apr. 28, 2023

(30) Foreign Application Priority Data
Oct. 29, 2020 (FR) FR2011099

(51) Int. Cl. A61F 9/008

(52) U.S. Cl. CPC A61F 9/0084; A61F 2009/0087; A61F 2009/00872; A61F 2009/00897

(57) ABSTRACT

The invention relates to a cutting apparatus including a femtosecond laser source (10) for emitting a Gaussian laser beam, a shaping system (30) including a spatial modulator of light for modulating the Gaussian laser beam, a sweeping optical scanner (40) for moving the modulated laser beam, an optical focusing system (50) for focusing the modulated laser beam, characterised in that the processing device further comprises a control unit (60) for controlling the femtosecond laser source (10), the shaping system (30), the sweeping optical scanner (40) and the optical focusing system (50), in order to produce: —at least one vertical cutting plane, and —at least one horizontal cutting plane; the spatial light modulator of the shaping system (30) being capable of emulating an axicon for generating a Bessel beam.

### TECHNICAL FIELD

The present invention relates to the technical field of surgical operations carried out with a femtosecond laser, and more particularly to that of ophthalmic surgery, in particular for applications of cutting corneas or lenses.

The invention relates to a device for cutting a human or animal tissue, such as a cornea or a lens, using a femtosecond laser source.

The term femtosecond laser source means a light source capable of emitting a laser beam in the form of ultra-short pulses, the duration of which is between 1 femtosecond and 100 picoseconds, preferably between 1 and 1000 femtoseconds, in particular in the order of a hundred femtoseconds.

### PRIOR ART

The femtosecond laser source is an instrument capable of cutting the corneal tissue, for example, by focusing a laser beam in the stroma of the cornea, and by producing a succession of adjacent small gas bubbles.

More precisely, during the focusing of the laser beam in the cornea, a plasma is generated by non-linear ionisation when the intensity of the laser exceeds a threshold value, named the optical breakdown threshold. A gas bubble then forms, causing a very localised disruption of the surrounding tissues. Hence, the volume actually ablated by the laser beam is very small compared to the disrupted zone.

The zone cut by the laser beam at each pulse is very small, in the micron or tens of micron range, according to the power and focusing of the beam. Hence, a lamellar corneal cut can only be obtained by producing a series of contiguous impacts over the entire surface of the zone to be cut.

An apparatus for cutting a (human or animal) ocular tissue 2 using a femtosecond laser source 1 is known from document WO 2016/055539. This cutting apparatus is illustrated in FIG. 1.

The cutting apparatus makes it possible, using a laser beam 11 from a femtosecond laser source 1, to generate a plurality of laser impact points simultaneously in a focal plane 101 of the cutting apparatus. As shown in FIG. 2, each impact point forms a respective gas bubble 102. In order to simultaneously generate a plurality of impact points, the cutting apparatus comprises a spatial light modulator 3 (SLM). A phase mask is applied to the SLM 3. This phase mask can modulate the phase of the wavefront of the laser beam 11 from the femtosecond laser source 1. The phase modulation of the wavefront makes it possible to delay or advance the phase of various points of the surface of the beam relative to the initial wavefront, in order that each of these points produces constructive interference at N distinct points in the focal plane 101 of the cutting apparatus. This redistribution of energy into a plurality of impact points only occurs in a single plane (i.e. the focal plane of the cutting apparatus) and not all along the propagation path of the modulated laser beam. Hence, the phase modulation of the wavefront makes it possible to generate a single modulated laser beam 31 which forms a plurality of impact points only in the focal plane 101: the modulated laser beam 31 is unique all along its propagation path.

In order to cut a lens on a surface of 1 mm², it is necessary to produce approximately 10,000 impact points, very close to one another. Generating a plurality of impact points simultaneously, reduces the time required in order to cut a lens surface by increasing the surface treated with a single laser firing and reducing the number of passages back-and-forth required to produce a plurality of lines of adjacent points.

The simultaneously generated plurality of impact points constitutes a pattern. By moving 103 this pattern in the focal plane 101 of the cutting apparatus, it is possible to form a horizontal cutting plane 104 containing a multitude of gas bubbles 102 (cf. FIG. 3). In order to move the pattern in the focal plane 101, the cutting apparatus comprises a scanning device 4, composed of driveable galvanometric mirrors and/or stages allowing the movement of optical elements, such as mirrors or lenses. This scanning device 4—positioned downstream of the SLM 3—can move the modulated laser beam 31 along a trajectory back-and-forth along a succession of segments constituting a movement path of the beam. A horizontal cutting plane 104 is thus formed, containing a multitude of gas bubbles 102 (cf. FIG. 4).

When the multitude of gas bubbles 102 has been formed in the focal plane 101 of the cutting apparatus, the portion of lens situated above the horizontal cutting plane can be detached from the portion of lens situated below the horizontal cutting plane, by unhooking the tissue bridges 105 existing between the gas bubbles 102 using a tool.

During cataract surgery, a stack 106 of horizontal cutting planes 104 is formed by moving the focal plane of the cutting apparatus (cf. FIG. 5). In order to move the focal plane 101, the cutting apparatus comprises an optical focusing device 5—positioned downstream of the scanning device 4—composed, in particular, of one or more motorised lenses in order to enable their movement in translation along the optical path of the modulated laser beam by the SLM 3 and deflected by the scanning device 4.

By moving the focal plane 101 into various positions along the optical path of the laser beam, and by repeating, for each position of the focal plane, the steps:
generating a pattern of impact points, and
moving the pattern of impact points,
   it is possible to obtain a stack 106 of horizontal cutting planes 104. The various slices of lens defined by these horizontal cutting planes 104 can then be separated from one another.

In addition to horizontal cutting planes 104, it is desirable to produce vertical cutting planes 107 in the lens. These vertical planes 107 are produced between two successive horizontal planes (producing a lower horizontal cutting plane 104a then producing vertical cutting planes 107 then producing an upper horizontal cutting plane 104b). This makes it possible to subdivide the lens C into cubes 108 which can be sucked by a suction cannula 109 during cataract surgery, for example (cf. FIG. 7), in contrast to current systems which require an ultrasonic phacoemulsifier.

Currently, a vertical cutting plane 107 is obtained by producing lines of gas bubbles superposed in the lens C. In order to produce a vertical cutting plane, the laser beam from the laser source is not phase modulated. With each pulse of the femtosecond laser source, a single impact point is formed. This impact point can produce a gas bubble. By moving the laser beam using the scanning device, it is possible to move the impact point in the focal plane of the cutting apparatus. This makes it possible to produce a succession of adjacent small gas bubbles, which then form a cutting line in the focal plane of the cutting apparatus. By moving the focal plane—using the focusing device—into various positions along the optical path of the laser beam, lines of gas bubbles can be superposed in order to obtain a vertical cutting plane.

Such a vertical cutting plane being produced “point-by-point”, the operation of forming the various vertical cutting planes is slow. Indeed, currently, the impact points are produced at an average speed of 300,000 impacts/second. The “point-by-point” cutting of a lens on a surface of approximately 65 mm², taking account of the time during which the laser stops the production of pulses at the segment end in order to allow the mirrors to be positioned on the following segment, requires on average 15 seconds.

In order to overcome this disadvantage, and starting from the cutting apparatus according to WO 2016/055539, the inventors have tried to produce vertical cutting planes by implementing the principle of demultiplication of the impact points from each pulse of the laser source. In particular, the inventors have determined a phase mask to apply to the SLM in order to generate a plurality of simultaneous impact points 110 at various depths Z1, Z2, Z3, using a single modulated laser beam (cf. FIG. 8). For example, using a pattern composed of three (four, five, etc.) impact points 110a, 110b, 110c generated simultaneously at various depths Z1, Z2, Z3, it is theoretically possible, by moving the pattern along a movement segment using the scanning device, to simultaneously generate three (four, five, etc.) superposed lines of gas bubbles, which correspondingly reduces the time required for forming a vertical cutting plane.

However, the inventors have discovered that the alignment of the simultaneously generated impact points 110 was not sufficient, so that the lines of gas bubbles were not perfectly superposed. This alignment defect makes detachment of the lens cubes difficult.

Document WO 2018/020144 describes an apparatus for cutting transparent dielectric or semiconductor material. The apparatus comprises:
a laser source generating a laser beam,
an optical Bessel beam generator device configured to transform a Gaussian intensity spatial distribution of the laser beam into a Bessel intensity spatial distribution of the laser beam, transverse to the optical axis in the focusing zone,
a passive optical system comprising a phase and/or amplitude mask configured to modify the Bessel spatial distribution of the laser beam transversely and/or longitudinally with respect to the optical axis in the zone.

Document US 2015/164689 describes a laser cutting device for a transparent material.

Document US 2019/314194 describes a laser system for capsulorhexis surgery comprising:
a laser source,
a beam guidance system,
a beam focusing device,
a beam coupler configured to redirect the focused laser cutting beam, and
a patient interface lens.

Document US 2017/128259 describes a cutting system for implementing a femto-fragmentation procedure on a tissue in the lens of an eye, which requires that a laser beam is directed to and focused on a focal point in the lens of the eye.

An aim of the present invention is to provide a solution to the problem of forming vertical cutting planes in an ocular tissue (such as a cornea or a lens) based on the cutting apparatus described in WO 2016/055539.

### DISCLOSURE OF THE INVENTION

For this purpose, the invention proposes a cutting apparatus for a human or animal tissue, said apparatus including a femtosecond laser source configured to emit a Gaussian laser beam in the form of pulses and a processing device of the Gaussian laser beam, the processing device being arranged downstream of the femtosecond laser source, the processing device comprising:
a shaping system positioned on the trajectory of the Gaussian laser beam, in order to modulate the phase of the wavefront of the Gaussian laser beam, the shaping system comprising a spatial light modulator (SLM) and being configured to produce a modulated laser beam from the Gaussian laser beam,
an sweeping optical scanner arranged downstream of the shaping system in order to move the modulated laser beam,
an optical focusing system downstream of the shaping system, for focusing the modulated laser beam in a focal plane of the cutting apparatus and for moving the focal plane of the cutting apparatus into a plurality of positions along an optical axis of propagation of the modulated laser beam,
   characterised in that the processing device further comprises a control unit for driving the femtosecond laser source, the shaping system, the sweeping optical scanner and the optical focusing system, in order to produce at least one vertical cutting plane extending parallel to the optical axis, the control unit being configured to:
apply, to the shaping system, an axiconic modulation instruction in order to produce a Bessel-type modulated laser beam from the Gaussian laser beam, said modulation instruction including a phase mask (314, 315) emulating an axicon applied on the spatial light modulator (SLM), said phase mask (314, 315) having a rotational symmetry about a central symmetry point, the grey level of each point of the phase mask varying according to the distance between said point and the central symmetry point, said Bessel-type modulated laser beam having an impact point which allows to generate an oblong gas bubble in the tissue and thus to cut it at a depth much greater than with a Gaussian beam;
drive the optical scanner in order to move the impact point of the Bessel-type modulated laser beam along an optical movement path in order to successively form a plurality of adjacent gas bubbles, said gas bubbles constituting the vertical cutting plane.

In the context of the present invention, “vertical cutting plane” means a plane situated in the tissue to be treated and extending parallel to an optical axis of propagation of the laser beam from the cutting apparatus. In the context of the present invention, “horizontal cutting plane” means a plane situated in the tissue to be treated and extending perpendicular to the optical axis of propagation of the laser beam from the cutting apparatus.

In the context of the present invention, “impact point” means a zone of the laser beam included in its focal plane, in which the intensity of said laser beam is sufficient to generate a gas bubble in a tissue.

In the context of the present invention, “adjacent impact points” mean two impact points arranged facing one another and not separated by another impact point.

The term “neighbouring impact points” mean two points of a group of adjacent points between which the distance is minimal.

In the context of the present invention, “pattern” means a plurality of laser impact points generated simultaneously in a focal plane of the cutting apparatus.

Hence, the invention makes it possible to modify the intensity profile of the laser beam in the cutting plane, in such a way as to improve the quality or the speed of the cutting, according to the chosen profile. This intensity profile modification is obtained by modulating the phase of the laser beam.

The optical phase modulation is carried out by means of a phase mask. The energy of the incident laser beam is conserved after modulation, and the shaping of the beam is performed by acting on its wavefront. The phase of an electromagnetic wave represents the instantaneous situation of the amplitude of an electromagnetic wave. The phase depends on both time and space. In the case of the spatial shaping of a laser beam, only phase variations in space are considered.

The wavefront is defined as the surface of points of a beam having an equivalent phase (i.e. the surface consisting of points for which the times of travel from the source having emitted the beam are equal). The modification of the spatial phase of a beam therefore includes the modification of its wavefront.

This technique makes it possible to perform the cutting operation quicker and more efficiently, because it uses a plurality of laser spots each producing a cut and according to a controlled profile.

In the context of the present invention, the phase modulation of the wavefront allows to generate a single modulated laser beam which forms a plurality of impact points in the cutting plane only. Hence, the modulated laser beam is unique all along the propagation path. The phase modulation of the wavefront allows to delay or advance the phase of various points of the surface of the beam relative to the initial wavefront, in order that each of these points produces constructive interference at N distinct points in the focal plane of a lens. This redistribution of energy into a plurality of impact points only occurs in a single plane (i.e. the focal plane) and not all along the propagation path of the modulated laser beam.

By contrast, document US 2010/0133246 proposes using an optical system based on the phase and able to subdivide a primary beam into a plurality of secondary beams having different propagation angles.

The modulation technique according to the invention (by generating a single modulated laser beam) allows to limit the risk of degradation in the quality of the cut surface. Indeed, if a portion of the single modulated laser beam is lost along the propagation path of the beam, the intensities of all the impact points of the pattern will be attenuated at the same time (conservation of homogeneity between the various impact points of the pattern) but no impact point will disappear in the cutting plane. By contrast, with the beam subdivision technique in US 2010/0133246, if a portion of the plurality of secondary beams is lost along the propagation path, then certain impact points of the pattern (corresponding to the impact points generated by the lost secondary beams) will be missing in the cutting plane, which substantially degrades the quality of the cut performed.

Preferred, but non-limiting, aspects of the cutting apparatus are the following:
the object focal plane of the focusing system can be positioned at a non-zero distance from the image focal plane of the shaping system, such that the impact point of the Bessel-type modulated laser beam comprises:
   a ring focused in the focal plane of the cutting apparatus,
   a line of concentration of the rays of the Bessel-type modulated laser beam extending outside of the focal plane of the cutting apparatus,
said line for forming the oblong gas bubble, the ring having an intensity less than the intensity of the line, not allowing the gas bubble to form;
the control unit can be programmed to drive the optical focusing system such that the focal plane of the cutting apparatus extends along the optical axis, above the desired position for the vertical cutting plane;
the control unit can be programmed to drive the optical focusing system, such that the focal plane of the cutting apparatus extends along the optical axis, below the desired position for the vertical cutting plane;
the control unit can be further configured to drive the femtosecond laser source, the shaping system, the sweeping optical scanner, and the optical focusing system, in order to produce at least one horizontal cutting plane extending perpendicular to the optical axis;
the cutting apparatus can be suitable for successively producing horizontal and vertical cutting planes so as to form cubes of tissue:
   the control unit driving the femtosecond laser source, the shaping system, the sweeping optical scanner and the optical focusing system, in order to produce an initial horizontal cutting plane, then
   the control unit driving the femtosecond laser source, the shaping system, the sweeping optical scanner and the optical focusing system, in order to produce at least one vertical cutting plane located above, along the optical axis, the initial horizontal cutting plane, then
   the control unit driving the femtosecond laser source, the shaping system, the sweeping optical scanner and the optical focusing system, in order to produce a final horizontal cutting plane above, along the optical axis, said and at least one vertical cutting plane;
in order to produce a horizontal cutting plane, the control unit can be configured to:
   apply a multipoint phase mask to the shaping system in order to produce a single multipoint modulated laser beam, the multipoint phase mask being calculated to distribute the energy of the multipoint modulated laser beam into at least two impact points in the focal plane of the cutting apparatus;
   control the movement of the focusing system in order to make the focal plane of the cutting apparatus coincide with the desired depth for the horizontal cutting plane;
   activate the femtosecond laser source; and
   drive the sweeping optical scanner in order to move the impact points of the single multipoint modulated laser beam along a movement path;
in order to produce a vertical cutting plane, the control unit (60) is configured to:
   apply a linear phase mask to the shaping system in order to produce a Bessel modulated laser beam;
   control the movement of the focusing system in order to position the focal plane of the cutting apparatus above or below the desired depth for the vertical cutting plane;
   activate the femtosecond laser source; and
   drive the optical scanner in order to move the impact point of the Bessel modulated laser beam along a movement path.

### BRIEF DESCRIPTION OF THE FIGURES

Other features and advantages will emerge from the description which is given below, by way of illustration and not being in any way limiting, with reference to the attached figures, in which:

FIG. 1 is a schematic representation of a cutting apparatus described in WO 2016/055539;

FIG. 2 is a schematic representation of gas bubbles created by impact points in a focal plane of the cutting apparatus of FIG. 1,

FIG. 3 is a schematic representation of gas bubbles created successively by moving the impact points in the focal plane of the cutting apparatus of FIG. 1,

FIG. 4 is a schematic representation of a horizontal cutting plane obtained using the cutting apparatus of FIG. 1,

FIG. 5 is a schematic representation of a stack of horizontal cutting planes obtained using the cutting apparatus of FIG. 1,

FIG. 6 is a schematic representation of horizontal and vertical cutting planes,

FIG. 7 is a schematic representation of an eye of a patient,

FIG. 8 is a schematic representation of impact points formed simultaneously using an SLM of the cutting apparatus of FIG. 1,

FIG. 9 is a schematic representation of a cutting apparatus according to the invention,

FIG. 10a is a Bessel-type beam image along a longitudinal profile,

FIG. 10b is a Bessel-type beam image along a transverse profile,

FIG. 11 is a schematic representation illustrating the focusing of a Bessel-type non-diffracting beam,

FIG. 12a is an image of a first phase mask that can emulate the behaviour of a negative axicon on an SLM of the cutting apparatus according to the invention,

FIG. 12b is an image of a second phase mask that can emulate the behaviour of a positive axicon on an SLM of the cutting apparatus according to the invention,

FIG. 13a is a schematic representation of a Bessel-type beam along a longitudinal profile,

FIG. 13b is a representation of a Bessel-type beam along a transverse profile,

FIG. 14 is a partial assembly diagram of the cutting device,

FIG. 15 is a schematic representation of a Bessel beam,

FIG. 16 is a schematic representation showing the forming of a vertical cutting plane using a Gaussian laser beam on the one hand and a Bessel laser beam on the other hand.

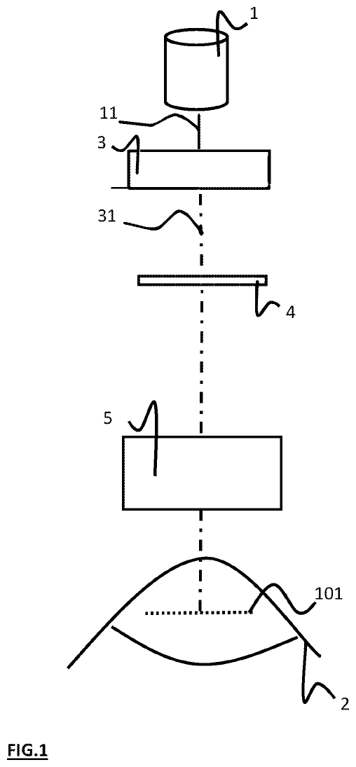

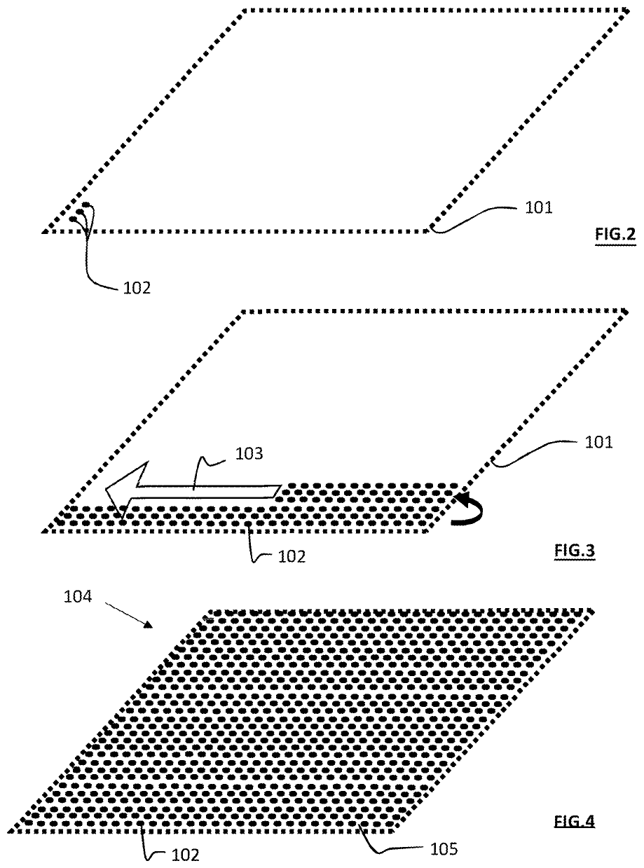

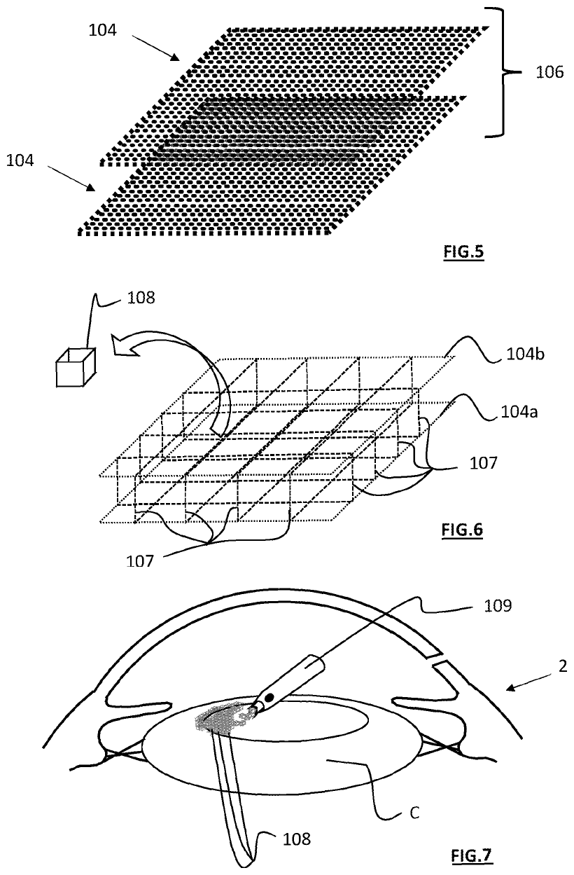

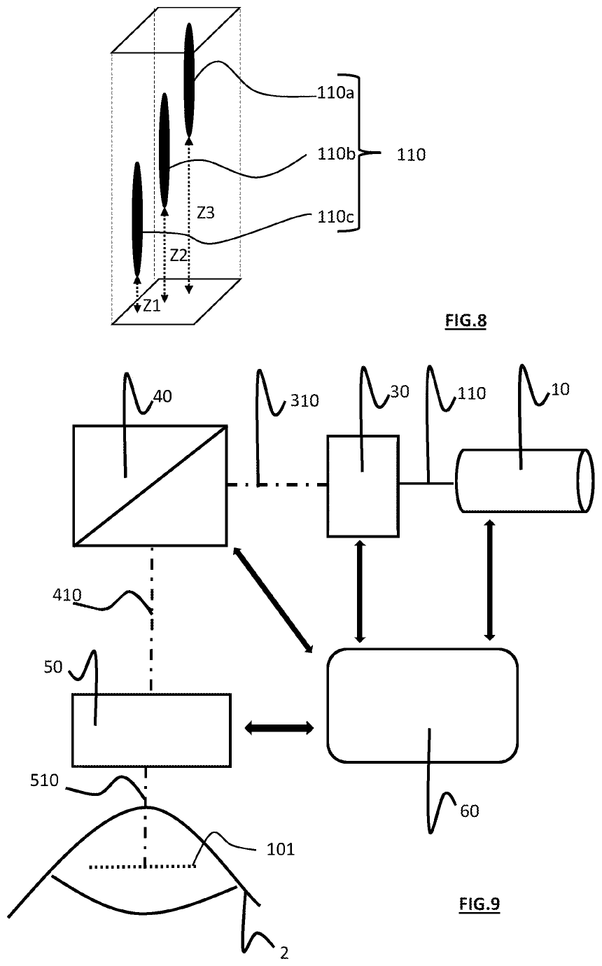

### DETAILED DISCLOSURE OF THE INVENTION

The invention relates to a system for cutting a human tissue by means of a femtosecond laser. In the remainder of the description, the invention will be described, by way of example, for the cutting of a lens of a human or animal eye.

### 1. Cutting Apparatus

With reference to FIG. 9, an embodiment is shown of the cutting apparatus according to the invention. This can be arranged between a femtosecond laser source 10 and a target to be treated 2.

The femtosecond laser source 10 is able to emit a Gaussian laser beam in the form of pulses. By way of example, the femtosecond laser source 10 emits light at a wavelength of 1030 nm, in the form of 400-femtosecond pulses. The femtosecond laser source 10 has a power of 20 W and a frequency of 500 kHz.

The target 2 is, for example, a human or animal tissue to be cut, such as a cornea or a lens.

The cutting apparatus comprises:
a shaping system 30 positioned on the trajectory of the laser beam 110 from the femtosecond laser 10,
an sweeping optical scanner 40 downstream of the shaping system 30,
an optical focusing system 50 downstream of the sweeping optical scanner 40, and
a control unit 60.

The shaping system 30 allows to modulate the phase of the laser beam 110 from the femtosecond laser source 10. This shaping system 30 is advantageously a programmable component.

The sweeping optical scanner 40 allows to orient the phase-modulated laser beam 310 from the shaping system 30 in order to move the cutting pattern along a movement path predefined by the user, in the focal plane 101 of the cutting system.

The optical focusing system 50 can move the focal plane 101, corresponding to the cutting plane, of the modulated and deflected laser beam 410.

The control unit 60 can drive the shaping system 30, the sweeping optical scanner 40 and the optical focusing system 50.

This cutting apparatus is suitable for forming horizontal and vertical cutting planes. Depending on the desired type of cutting plane (vertical or horizontal), the control unit 60:
configures the shaping system in order to modulate the laser beam 110 according to the desired aspect for the impact points; and
controls the sweeping optical scanner 40 and the optical focusing system 50 in order to generate the desired cutting plane.

As will be described in more detail below, the inventors have developed an original configuration solution of the cutting apparatus for forming vertical cutting planes.

### 2. Cutting Apparatus Elements

2.1. Shaping System

The spatial shaping system 30 of the laser beam can vary the wave surface of the laser beam 110 according to the desired shape for the point or impact points of the modulated laser beam.

The shaping system 30 preferably comprises a spatial light modulator, known by the acronym SLM.

The SLM allows to modulate the final energy distribution of the laser beam 110 from the laser source 10. The SLM is a device consisting of a layer of liquid crystals with controlled orientation, able to dynamically shape the wavefront, and therefore the phase of the laser beam 110. The layer of liquid crystals of an SLM is organised as a grid (or matrix) of pixels. The optical thickness of each pixel is electrically controlled by orientation of the liquid-crystal molecules belonging to the surface corresponding to the pixel. The SLM exploits the principle of liquid-crystal anisotropy, in other words the modification of the liquid-crystal index, as a function of their spatial orientation. The liquid crystals can be oriented using an electric field. Hence, the modification of the liquid-crystal index modifies the wavefront of the laser beam.

In a known manner, the SLM uses a phase mask, in other words a map determining how the phase of the laser beam 110 must be modified in order to obtain a given amplitude distribution. The phase mask is a two-dimensional image, each point of which is associated with a respective pixel of the SLM. This phase mask can drive the index of each liquid crystal of the SLM by converting the value associated with each point of the mask—represented as a level of grey between 0 and 255 (thus from black to white)—in a control value—represented in a phase between 0 and 27. Hence, the phase mask is a modulation instruction displayed on the SLM in order to cause, on reflection, an unequal spatial phase shift of the laser beam 110 illuminating the SLM. Of course, a person skilled in the art will appreciate that the range of grey level can vary according to the model of SLM used. For example, in certain cases, the grey level range can be between 0 and 220.

Different phase masks can be applied to the SLM depending on the type of cutting plane that the user wishes to produce, namely:
either a vertical cutting plane,
or a horizontal cutting plane.

In order to produce a vertical cutting plane, the phase mask used (hereafter referred to as the “linear phase mask”) makes it possible to apply a linear phase modulation with rotational symmetry. A Bessel-type modulated laser beam is thus obtained.

In order to produce a horizontal cutting plane, the phase mask used (hereinafter referred to as the “multipoint phase mask”) makes it possible to apply a phase modulation in order to distribute the energy of the laser beam into at least two impact points forming a pattern in the focal plane of the cutting system. A multipoint modulated laser beam is thus obtained.

2.1.1. Vertical Cutting Plane

With regard to the vertical cutting plane, the inventors propose modulating the phase of the laser beam 110 from the femtosecond laser source 10 so as to produce, downstream of the shaping system 30, a Bessel-type non-diffracting modulated laser beam 310.

A Bessel beam is referred to as “non-diffracting” because it has the property of maintaining a constant profile along the optical axis of propagation of the laser beam (hereafter referred to as the “optical axis”), contrary to the behaviour of a Gaussian laser beam (such as the laser beam 110 from the femtosecond laser source 10) which disperses when it is focused.

2.1.1.1. Bessel Beam

A perfect zero-order Bessel beam can be defined mathematically as a beam for which the electric field (E) is formally described by the zero-order Bessel function of first kind J₀:

E(r,ϕ,z)A₀J₀(kᵣr)e^{jk}z^{z}

where:
A₀ is the amplitude of the electric field,
k_{z} and kᵣ are the longitudinal and radial wave vectors,
z, r, and ϕ are the longitudinal, radial and azimuthal components.

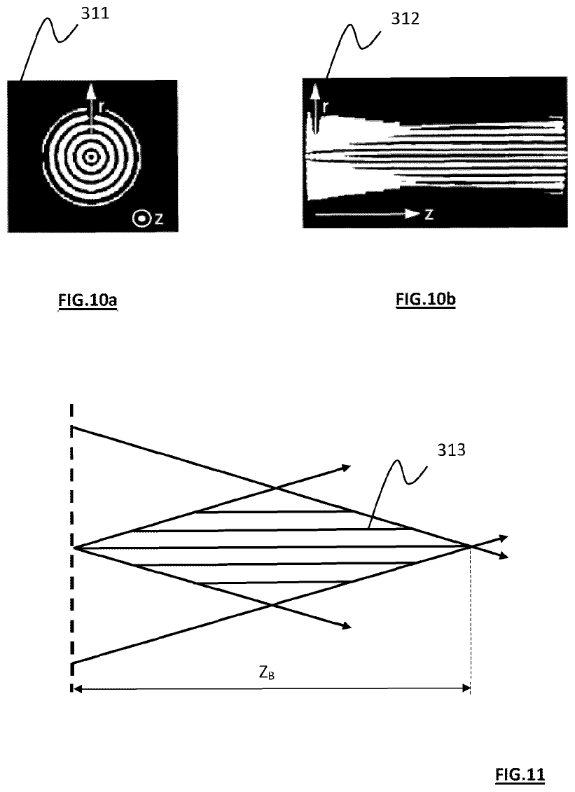

The profile of the Bessel beam is represented by a central peak of maximum intensity surrounded by concentric rings of lower intensity, as illustrated in FIGS. 10a and 10b which are respectively face and side views of a Bessel beam relative to its optical axis.

FIGS. 10a and 10b show a propagation with a constant profile over a distance of approximately 100 μm (image 312, FIG. 10b) with a diameter of the focusing spot (image 311, FIG. 10a) less than 1 μm. In comparison, a Gaussian beam generally has a constant propagation profile over 20 μm with a focusing spot diameter of 1 μm.

With reference to FIG. 11, the forming of the Bessel beam 313 results from the interference of plane waves, the wave vectors of which form a conical surface.

In theory, the transverse extension of the annular structure is infinite, as well as the non-diffractive propagation distance.

In practice, the experimental Bessel beam has a finite non-diffractive propagation distance Z_{B} along the optical axis, due to the finite propagation observed optically and the limited quantity of energy. This finite non-diffractive propagation distance Z_{B} defines a non-diffraction zone ZND.

It is assumed that Z_{B}>>Z_{R}, Z_{R} being the Rayleigh distance of the usual Gaussian beam of similar transverse size. In other words, the depth (i.e. dimension in a direction parallel to the optical axis of propagation of the laser beam) of each impact point of a Bessel beam is much greater than the depth of each impact point with a Gaussian laser beam (such as the laser beam from the femtosecond laser source).

Hence, the use of a Bessel beam can cut a much larger depth of tissue then with a Gaussian beam. In particular, using a single impact point of a Bessel beam, it is possible to cut a tissue to a depth equivalent to that of four superposed impact points of a Gaussian beam. The movement, by the sweeping optical scanner, of the impact point of a Bessel beam can generate a vertical cutting plane that is perfectly vertical, four times more rapidly than with a Gaussian beam impact point.

Due to its specific formation based on a conical wavefront, the Bessel beam has remarkable self-regenerating properties, which means that the beam can regenerate itself within the non-diffraction zone ZND after any obstacle on its path. This can ensure the quality of the cutting of the vertical planes by guaranteeing the formation of an extended gas bubble at each firing of the laser source 10, even when a part of the modulated laser beam 310 is masked by an obstacle.

The generation of a plurality of impact points at different depths using a multipoint modulated laser beam does not make it possible to obtain a vertical cutting plane of quality equivalent to that of a vertical cutting plane obtained using a Bessel beam. Indeed, with a multipoint modulated laser beam enabling the generation of a plurality of impact points along the optical axis, imperfections in the phase modulation generate a light that is not controlled at the level of a focal plane of the optical focusing system. This uncontrolled light interferes with the desired pattern of impact points. It is therefore impossible to precisely control the relative intensities of the impact points in the case of a multipoint modulated laser beam enabling the generation of a plurality of impact points along the optical axis.

Hence, due to the self-regenerating capacities of the Bessel beam, the impact point from a Bessel beam has an important advantage with respect to the simultaneous impact points formed along the optical axis by a multipoint modulated laser beam.

2.1.1.2. Linear Phase Mask for Forming a Bessel-Type Modulated Laser Beam

There are various techniques for generating a Bessel beam using a Gaussian laser beam. These techniques generally involve an axiconic phase modulation.

In particular, the Bessel beam can be obtained by using a conical lens known as an “axicon”. The conical lens can be concave/hollow (referred to as a “negative axicon”) or convex/domed (referred to as a “positive axicon”).

The inventors propose using the shaping system 30 including the SLM for generating the Bessel beam in order to avoid the use of an optical/mechanical element. To this effect, a linear phase mask (able to emulate an axicon) is applied to the SLM by the control unit 60. The SLM then enables a conical phase modulation of the Gaussian laser beam 110 from the femtosecond laser source 10. Hence, by using the same SLM, it becomes possible to produce a horizontal multipoint cutting plane, then vertical cutting planes in Bessel beam mode without changing the optical elements and therefore considerably reducing the time of the surgical procedure, compatible with an application on the eyeball of the patient of less than 3 minutes.

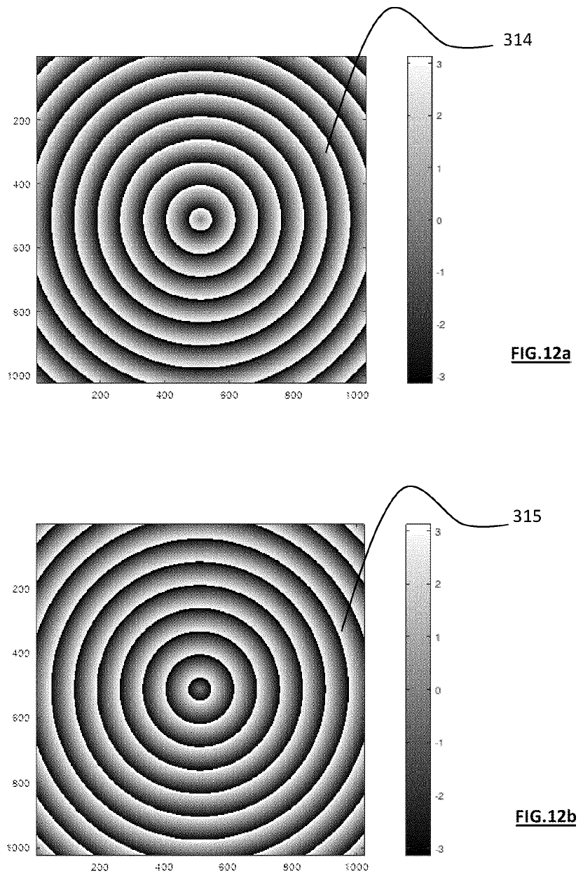

Two examples of such phase masks are shown in FIGS. 12a and 12b. When one of the first and second phase masks is applied to the SLM, the SLM is capable of printing the phase profile of an axicon on the input Gaussian laser beam 110 in order to obtain a Bessel-type modulated laser beam 310 at the output of the shaping system 30.

With reference to FIG. 12a, the first linear phase mask (reference 314) allows to emulate the behaviour of a negative axicon (i.e. concave axicon). With reference to FIG. 12b, the second linear phase mask (reference 315) allows to emulate the behaviour of a positive axicon (i.e. convex axicon). These first and second phase masks each have a rotational symmetry around a central symmetry point, the grey level of each pixel varying depending on the distance between said pixel and the central symmetry point.

When one of the phase masks shown in FIGS. 12a and 12b is applied to the SLM, the shaping system 30 can form a Bessel-type modulated laser beam 310 (at the outlet of the shaping system 30) from the Gaussian laser beam 110 from the femtosecond laser source 10 (at the inlet of the shaping system 30). Thus, a modulated laser beam is obtained having a Bessel beam spatial intensity distribution.

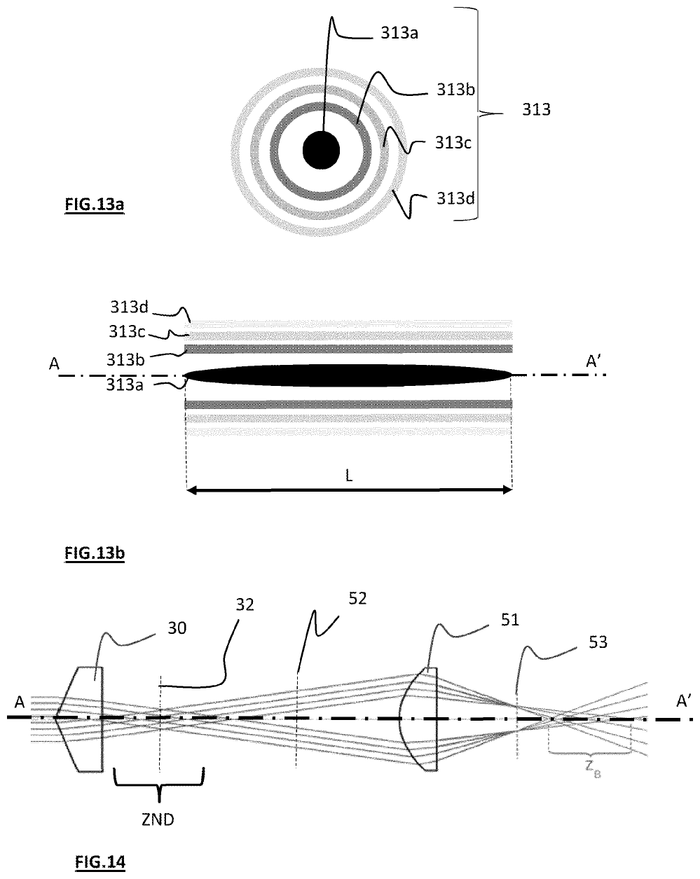

With reference to FIGS. 13a and 13b, this Bessel-type modulated laser beam comprises, in a plane transverse to the optical axis:
a central spot 313a of maximum intensity, and
a plurality of concentric rings 313b, 313c, 313d of decreasing intensity as a function of the radial distance to the optical axis.

This Bessel beam 313 extends over a depth L along the optical axis A-A′ (i.e. in the non-diffraction zone ZND of the Bessel beam). The choice of grey level values for the points of the linear phase mask can optimise the depth L of the Bessel beam 313 and therefore the volume within which it energy is deposited.

The linear phase mask to be applied to the SLM of the shaping system for forming a Bessel modulated laser beam can be calculated:
using a partitioning algorithm (Vellekoop and Mosk, 2008),
or any other algorithm known to a person skilled in the art.

2.1.1.3. Assembly of the Cutting Apparatus in the Context of the Cutting of a Tissue Using a Bessel-Type Modulated Laser Beam

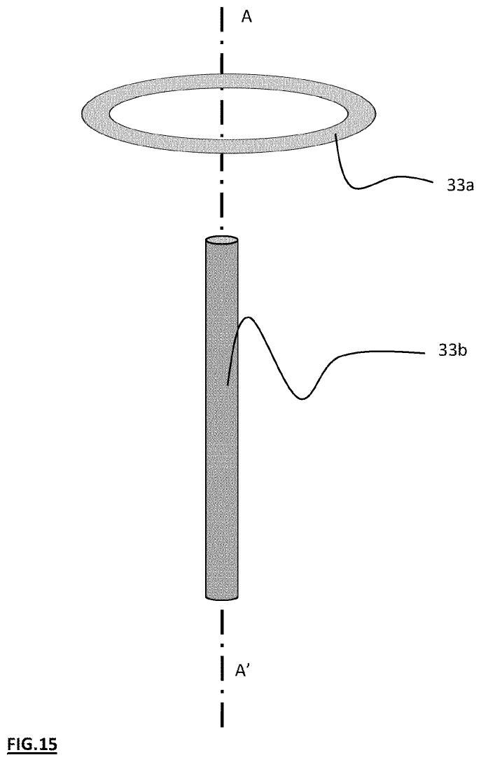

FIG. 14 shows an assembly diagram of the cutting apparatus. This assembly diagram is partial, in that it does not show the femtosecond laser source and the sweeping optical scanner. Furthermore, in FIG. 14, the optical focusing system 50 (in its entirety) is represented by an equivalent lens 51, it being well-known to a person skilled in the art that the optical focusing system 50 does not only consist of a fixed lens.

With reference to FIG. 14, the Bessel beam 313 is formed just after the conical phase modulation plane, in other words just after the SLM of the shaping system 30. The SLM simulating a conical lens (negative or positive axicon), the central spot 313a of maximum intensity of the Bessel beam forms in the image focal plane 32 of the SLM.

The equivalent lens 51 of the optical focusing system 50 is arranged downstream of the shaping system 30, and is arranged so that the object focal plane 52 of the equivalent lens extends at a non-zero distance from the image focal plane 32 of the shaping system 30 along the optical axis.

Hence, the object focal plane 52 of the equivalent lens 51 of the optical focusing system 50 extends outside of the non-diffraction zone ZND of the Bessel beam, so that at the outlet of the cutting system, an impact point as illustrated in FIG. 15 is obtained. This impact point consists of:
a Bessel ring 33a focused in the image focal plane 53 of the equivalent lens 51 (corresponding to the focal plane of the cutting apparatus),
a line 33b of concentration of the rays of the Bessel beam—corresponding to the image of the non-diffraction zone ZND—said line 33b forming outside the image focal plane 53 of the equivalent lens 51.

In the context of the present invention, this is the concentration line 33b of the impact point which is used to produce the vertical cutting plane (the energy contained in the Bessel ring is not sufficient to form a gas bubble).

The line 33b of concentration of rays can be formed either before or after the ring 33a, depending on the sign of the phase modulation. In other words, the position of the line 33b relative to the ring 33a depends on the type of axicon (positive or negative) that is emulated using the linear phase mask.

Since the Bessel non-diffraction zone ZND (i.e. the line 33b) is moved outside the focal plane of the cutting system, no interference is produced with the non-modulated light. This allows a better control the intensity profile without energy losses linked to the filtering of the beam.

2.1.2. Horizontal Cutting Plane

With regard to the cutting of a horizontal plane, the inventors propose modulating the phase of the laser beam 110 from the femtosecond laser source 10 so as to produce, downstream of the shaping system 30, a multipoint modulated laser beam.

For this purpose, a multipoint phase mask to be applied to the SLM in order to obtain the multipoint modulated laser beam is calculated. The multipoint phase mask is generally calculated by:
an iterative algorithm based on the Fourier transform, such as an Iterative Fourier Transform Algorithm (IFTA),
various optimisation algorithms, such as genetic algorithms, or simulated annealing.

This multipoint phase mask is calculated in order to form intensity peaks in the focal plane of the cutting apparatus, each intensity peak producing a respective impact point in the focal plane of the cutting apparatus.

More precisely, the multipoint phase mask is calculated in order to distribute the energy of the laser beam from the laser source into a plurality of impact points in the focal plane of the cutting apparatus. This modulation of the wavefront can be seen as a two-dimensional interference phenomenon. Each portion of the initial laser beam from the source is delayed or advanced with respect to the initial wavefront in order that each of these portions is redirected so as to produce constructive interference at N distinct points in the focal plane of a lens. This redistribution of energy into a plurality of impact points only occurs in a single plane (i.e. the focal plane) and not all along the propagation path of the modulated laser beam. Hence, the multipoint laser beam obtained (at the outlet of the shaping system 30) is unique: the observation of the modulated laser beam before or after the focal plane of the cutting apparatus (corresponding to the focal plane of the optical focusing system 50) does not make it possible to identify a redistribution of the energy into a plurality of distinct impact points, due to this phenomenon that can be assimilated to constructive interferences (which only take place in a plane and not throughout the propagation as in the case of the separation of an initial laser beam into a plurality of secondary laser beams).

The fact of having a single multipoint modulated laser beam facilitates the incorporation of a scanning system—such as an optical scanner—in order to move the plurality of impact points in the focal plane. Indeed, the inlet diameter of a scanning system being of the order of the diameter of the initial laser beam from the laser source 10, the use of a single multipoint modulated laser beam (the diameter of which is substantially equal to the diameter of the initial laser beam) limits the risk of aberration which can be produced with the technique of beam subdivision, such as described in US 2010/0133246.

The shaping system 30 therefore makes it possible, using a Gaussian laser beam generating a single impact point, and by means of the multipoint phase mask applied to the SLM, to distribute its energy by phase modulation so as to simultaneously generate a plurality of impact points in the focal plane of the cutting apparatus, using a single laser beam shaped by phase modulation (a single beam upstream and downstream of the SLM). This can reduce the time required to produce a horizontal cutting plane.

For example, in the case of a multipoint modulated laser beam having three impact points, the time required to produce a horizontal cutting plane is reduced by a factor of six (relative to the production of the same horizontal cutting plane using a Gaussian laser beam generating a single impact point). A person skilled in the art knows to calculate a value at each point of the multipoint phase mask in order to distribute the energy of the laser beam into different impact points in the focal plane of the cutting apparatus.

2.2. Sweeping Optical Scanner

The sweeping optical scanner 40 allows to deflect the modulated laser beam (Bessel or multipoint) 310 so as to move the point or impact points into a plurality of positions 43a-43c in the cutting plane.

The sweeping optical scanner 4 comprises:
an inlet orifice for receiving the phase-modulated laser beam 31 from the shaping unit 30,
one or more optical mirrors pivoting about at least two axes in order to deflect the phase-modulated laser beam 310, and
an outlet orifice for sending the deflected modulated laser beam 410 to the optical focusing system 50.

The optical scanner 4 used is, for example, an IntelliScan III scan head from SCANLAB AG.

The inlet and outlet orifices of such an optical scanner 40 have a diameter of between around 10 to 20 millimetres, and the attainable scanning speeds are in the range of about 1 m/s to 10 m/s.

The one or more mirrors are connected to one or more motors to enable their pivoting. This one or more motors for pivoting the one or more mirrors are advantageously driven by the control unit 60 which will be described in more detail below.

The control unit 60 is programmed to drive the sweeping optical scanner 40 so as to move the point or impact points along a movement path contained in the cutting plane.

In the case of a vertical cutting plane, the movement path comprises a segment. In this case, the control unit 60 can be configured to order the optical scanner 40 to make a back-and-forth movement of the Bessel impact point in order to cut the cutting plane over its entire depth. For example, if the optical scanner 40 starts the segment from the left, on the way back it will start this segment from the right, then from the left, then from the right and so on over the entire height of the cutting plane.

In the case of a horizontal cutting plane, the movement path comprises a plurality of cutting segments. The movement path can advantageously have a slot shape.

Advantageously, the control unit 6 can be programmed to activate the femtosecond laser 10 when the scanning speed of the optical scanner 40 is greater than a threshold value. This can synchronise the emission of the laser beam 110 with the scanning of the sweeping optical scanner 40. More precisely, the control unit 60 activates the femtosecond laser 10 when the pivoting speed of the one or more mirrors of the optical scanner 40 is constant. This makes it possible to improve the cutting quality by producing a homogeneous surface of the cutting plane.

2.3. Optical Focusing System

The optical focusing system 50 allows to move the focal plane of the cutting apparatus according to the type of cutting plane to be performed.

The optical focusing system 50 comprises:
an inlet orifice for receiving the phase-modulated and deflected laser beam from the sweeping optical scanner 40,
one or more motorised lenses for enabling their movement in translation along the optical path of the modulated and deflected laser beam, and
an outlet orifice for sending the focused laser beam to the tissue to be treated.

The one or more lenses are used with the optical focusing system 50 can be f-theta lenses or telecentric lenses. The f-theta and telecentric lenses make it possible to obtain a focusing plane over the entire field XY, in contrast to standard lenses for which it is curved. This can guarantee a constant focused beam size over the entire field. For f-theta lenses, the position of the beam is directly proportional to the angle applied by the scanner, whereas the beam is always normal to the sample for telecentric lenses.

The control unit 60 is programmed to drive the movement of the lens or lenses of the optical focusing system 50 so as to move the focal plane of the cutting apparatus depending on the type of cutting plane to be produced.

In the case of a horizontal cutting plane, the cutting plane corresponds to the focal plane of the cutting apparatus. The control unit 60 drives the movement of the lens or lenses of the optical focusing system 50 in order to focus the modulated and deflected laser beam 410 at a desired depth corresponding to the depth of the cutting plane to be produced.

In the case of a vertical cutting plane, the cutting plane can be situated:
below the focal plane of the cutting apparatus in the case where the linear phase mask used makes it possible for the SLM to emulate a positive axicon (the Bessel ring 33a is situated above the concentration line 33b used to produce the cutting), in this case the control unit 60 drives the optical focusing system 50 in order to focus the modulated and deflected laser beam 410 at a desired depth greater than the depth of the cutting plane to be produced (in order that the line of concentration 33b of the impact point is located at the depth of the cutting plane to be produced),
above the focal plane of the cutting apparatus in the case where the linear phase mask used makes it possible for the SLM to emulate a negative axicon (the Bessel ring 33a is situated below the concentration line 33b used to produce the cutting), in this case the control unit 60 drives the optical focusing system 50 in order to focus the modulated and deflected laser beam 410 at a desired depth less than the depth of the cutting plane to be produced (in order that the line of concentration 33b of the impact point is located at the depth of the cutting plane to be produced).

Finally, the control unit 6 can be programmed to drive the sweeping optical scanner 4 so as to vary the area in the focusing plane 21 between two successive cutting planes 22d, 22e. This makes it possible to vary the shape of the finally cut volume 23 depending on the targeted application.

Preferably, the distance between two successive cutting planes is between 2 μm and 500 μm, and in particular:
between 2 and 20 μm, in order to treat a volume requiring a high precision, for example in refractive surgery, preferably with a spacing between 5 and 10 μm, or
between 20 and 500 μm, in order to treat a volume not requiring a high precision, for example in order to destroy the central part of a lens nucleus, preferably with a spacing between 50 and 300 μm.

Of course, this distance can vary in a volume 23 consisting of a stack of cutting planes 22a-22e.

2.4. Control Unit

As previously indicated, the control unit 60 allows to control the various elements constituting the cutting apparatus, namely the femtosecond laser source 10, the shaping system 30, the sweeping optical scanner 40 and the optical focusing system 50.

The control unit 60 is connected to these different elements by means of one or more communication buses enabling:
transmission of control signals such as
   the activation signal to the femtosecond laser source 10,
   the phase mask to the shaping system 30,
   the scanning speed to the sweeping optical scanner 40,
   the position of the sweeping optical scanner 40 along the movement path,
   the cutting depth to the optical focusing system 50.
the reception of measurement data from the various elements of the system, such as
   the scanning speed reached by the sweeping optical scanner, or
   the position of the optical focusing system, etc.

The control unit 60 can be composed of one or more workstations, and/or one or more computers or can be of any other type known to a person skilled in the art. The control unit 60 can, for example, comprise a mobile telephone, an electronic tablet (such as an IPAD®), a personal digital assistant (PDA), etc.

In all cases, the control unit 60 comprises a processor programmed to enable the driving of the femtosecond laser source 10, the shaping system 30, the sweeping optical scanner 40, the optical focusing system 50, etc.

Advantageously, the control unit 60 is programmed to vary the shape of the modulated laser beam between two successive cutting planes, in particular between a horizontal cutting plane and a vertical cutting plane.

2.5. Principle of Operation

The operating principle of the cutting apparatus will now be described in more detail with reference to the destruction of a lens in the context of a cataract operation.

In order to partition the lens into cubes that can be sucked by a suction cannula, horizontal and vertical cutting planes are formed, starting with the deepest horizontal cutting plane in the lens and stacking the successive vertical and horizontal cutting planes up to the horizontal cutting plane closest to the surface of the lens.

In a first step, the deepest horizontal cutting plane is produced. The control unit 60:
applies a multipoint phase mask to the shaping system 30 in order to produce a multipoint modulated laser beam,
controls the movement of the focusing system 50 in order to make the focal plane of the cutting apparatus coincide with the deepest desired cutting plane,
activates the femtosecond laser source 10, and
drives the movement of the sweeping optical scanner along the optical path (for example in a slot).

A succession of firings are produced in the focal plane of the cutting apparatus. At each firing, a plurality of impact points simultaneously focus in the focal plane. Each impact point forms a gas bubble. The optical scanner can move the plurality of impact points in the focal plane between each firing. When the entire surface of the horizontal cutting plane is covered with gas bubbles, the horizontal cutting plane is finalised.

In a second step, a plurality of adjacent vertical cutting planes are then produced with the cutting apparatus. For each vertical cutting plane, the control unit 60:
applies a linear phase mask to the shaping system 30 in order to produce a Bessel modulated laser beam,
controls the movement of the focusing system 50 in order to position the concentration line 33b of the impact point in the cutting plane (the focusing plane being above or below the cutting plane depending on the emulated axicon on the shaping system being either a positive or negative axicon),
activates the femtosecond laser source 10, and
drives the movement of the sweeping optical scanner along the optical path (for example in a segment).

A succession of firings are performed. An impact point is formed at each firing, this impact point including:
a ring 33a of low-intensity situated in the focal plane of the cutting apparatus,
a concentration line 33b of high-intensity situated on or under the focal plane of the cutting apparatus.

Each impact point forms an oblong gas bubble along the optical axis of propagation of the modulated laser beam. The optical scanner allows to move the impact point under/over the focal plane between each firing. When the entire movement path is covered with gas bubbles, the vertical cutting plane is finalised.

If the depth of the concentration line of 33b is less than the desired depth for the vertical cutting plane, then the control unit 60 can control the sweeping optical scanner 40 and the optical focusing system 50 in order to move the impact point back and forth along the optical path by varying the depth of the focal plane of the cutting apparatus between the back and forth movement.

Thus a plurality of vertical cutting planes are obtained above the initial horizontal cutting plane.

In a third step, an upper horizontal plane is produced in order to cap the vertical cutting planes. This horizontal cutting plane is produced according to the same method as that described with reference to the first step.

Thus, cubes of lens are obtained, defined between the horizontal and vertical planes produced in the first, second and third steps.

These steps can be reiterated in order to produce a stack of lens cubes.

### 3. Conclusions

### 3.1. Advantages Associated with the Use of a Bessel Beam

As previously indicated, it is possible to cut a much larger depth of tissue with a Bessel beam, which makes it possible to generate a cutting plane much more quickly than with a Gaussian beam.

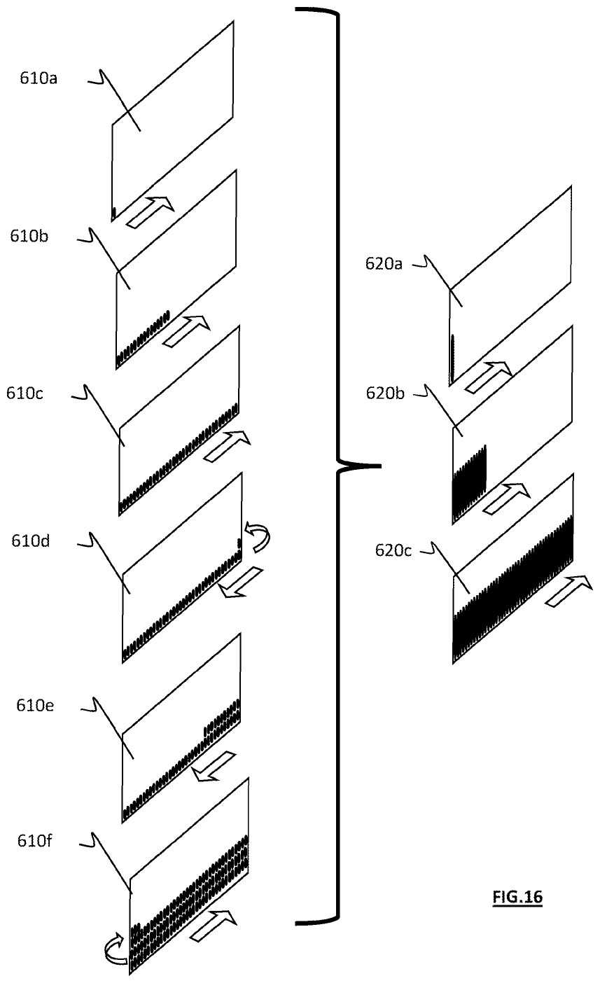

By way of indication, FIG. 16 compares the time required for producing a vertical cutting plane:
using a Gaussian beam on the one hand (images 610a to 610f),
using a Bessel beam on the other hand (images 620a to 620c).

In the case of the use of a Gaussian beam generating a single impact point moved by the sweeping optical scanner, it is necessary to perform four back-and-forth movements in order to form the gas bubbles which are superposed in order to constitute the cutting plane. The time required for producing the vertical cutting plane can be formulated as follows:

T1=(8×t1)+(7×t2)

Where:
T1 corresponds to the total cutting time
t1 corresponds to the time for travelling along one line
t2 corresponds to the time for producing a U-turn.

By assuming t1≈t2=t, then the cutting time of the plane is equal to 15 tin the case of a Gaussian beam.

In the case of the use of a Bessel beam, only one back-and-forth movement is necessary in order to constitute the cutting plane. The time required for producing the vertical cutting plane can be formulated as follows:

T2=(2×t1)+(1×t2)

Where:
T2 corresponds to the total cutting time
t1 corresponds to the time for travelling along one line
t2 corresponds to the time for producing a U-turn.

By assuming t1≈t2=t, then the cutting time of the plane is equal to 3t in the case of a Bessel beam.

The use of an SLM for shaping a Gaussian beam according to an axiconic modulation instruction for obtaining a Bessel modulated laser beam generating an oblong impact point, therefore allows to reduce the time necessary for producing a vertical cutting plane by a factor of 5.

### 3.2. General Conclusion

Hence, the invention makes it possible to have an effective three-dimensional cutting tool, contrary to current tools which can only produce two-dimensional cutting planes (vertical single spot cuts, in quarters or rods, without the possibility of combining them with horizontal cuts in an acceptable time).

In particular, the cutting apparatus is configured to carry out a surgical cutting operation in a rapid and efficient manner. The SLM can dynamically shape the wavefront of the laser beam from the femtosecond laser source, since it can be digitally parametrised:
the horizontal cutting planes are produced using a multipoint phase mask,
the vertical cutting planes are produced using a linear phase mask.

The phase mask change being produced in several milliseconds, the sequence of successively horizontal and then vertical and so on cutting planes is made extremely quickly without having to mobilise optical/mechanical elements, which gives this invention its unique character enabling a lens to be cut into 10,000 to 20,000 cubes in a time of around 30 seconds, whereas it would require between 5 and 10 minutes with current systems in order to perform the equivalent, which is of course unacceptable from the point of view of the comfort and safety of the patient.

The invention has been described for cutting operations of a lens in the field of ophthalmic surgery, but it is obvious that it can be used for other types of ophthalmic surgery operations without going beyond the scope of the invention. For example, the invention has an application in corneal refractive surgery, such as the treatment of ametropias, in particular myopia, hypermetropia, astigmatism, and in the treatment of the loss of accommodation, in particular presbyopia.

The invention also has an application in the treatment of cataracts with incision of the cornea, cutting of the anterior lens capsule, and fragmentation of the lens. Finally, more generally, the invention relates to all clinical or experimental applications on the cornea or the lens of a human or animal eye.

Still more generally, the invention relates to the broad field of laser surgery and has advantageous application when cutting is involved, and more particularly vaporising of human or animal soft tissues with a high water content.

The reader will understand that many modifications can be made to the above-described invention without materially departing from the novel teachings and advantages described here.

1. A cutting apparatus for cutting a tissue, wherein said apparatus includes a femtosecond laser source configured to emit a Gaussian laser beam in the form of pulses, and a processing device of the Gaussian laser beam, wherein the processing device is arranged downstream of the femtosecond laser source and comprises:
a shaping system positioned on the trajectory of the Gaussian laser beam, wherein the shaping system comprises a spatial light modulator which modulates the phase of the wavefront of the Gaussian laser beam to produce a modulated laser beam,
an sweeping optical scanner arranged downstream of the shaping system which moves the modulated laser beam,
an optical focusing system downstream of the shaping system which focusses the modulated laser beam in a focal plane of the cutting apparatus and which is configured to move the focal plane of the cutting apparatus into a plurality of positions along an optical axis of propagation of the modulated laser beam,
wherein the processing device further comprises a control unit which drives the femtosecond laser source, the shaping system, the sweeping optical scanner and the optical focusing system, in order to produce at least one vertical cutting plane extending parallel to the optical axis,
and wherein the control unit is configured to:
apply, to the shaping system, an axiconic modulation instruction in order to produce a Bessel-type modulated laser beam from the Gaussian laser beam, said modulation instruction including a phase mask which emulates an axicon applied on the spatial light modulator, wherein said phase mask has a rotational symmetry about a central symmetry point, and wherein the grey level of each point of the phase mask varies according to the distance between said point and the central symmetry point, said Bessel-type modulated laser beam having an impact point which enables an oblong gas bubble to be generated in the tissue and thus cutting at a depth much greater than a Gaussian beam;
drive the sweeping optical scanner in order to move the impact point of the Bessel-type modulated laser beam along an optical movement path such that adjacent oblong gas bubbles are successively formed, wherein said gas bubbles constitutes the vertical cutting plane.

2. The cutting apparatus according to claim 1, wherein the object focal plane of the focusing system is positioned at a non-zero distance from the image focal plane of the shaping system, such that the impact point of the Bessel-type modulated laser beam includes:
a ring focused in the focal plane of the cutting apparatus,
a line of concentration of the rays of the Bessel-type modulated laser beam extending outside the focal plane of the cutting apparatus,
wherein said line forms the oblong gas bubble, and wherein the ring has an intensity less than the intensity of the line, not allowing gas bubble formation.

3. The cutting apparatus according to claim 1, wherein the control unit is programmed to drive the optical focusing system such that the focal plane of the cutting apparatus extends along the optical axis, above the desired position for the vertical cutting plane.

4. The cutting apparatus according to claim 1, wherein the control unit is programmed to drive the optical focusing system such that the focal plane of the cutting apparatus extends along the optical axis, below the desired position for the vertical cutting plane.

5. The cutting apparatus according to claim 1, wherein the control unit is further configured to drive the femtosecond laser source, the shaping system, the sweeping optical scanner and the optical focusing system, in order to produce at least one horizontal cutting plane extending perpendicular to the optical axis.

6. The cutting apparatus according to claim 5, which is configured to successively produces horizontal and vertical cutting planes in order to form cubes of tissue, wherein:
the control unit drives the femtosecond laser source, the shaping system, the sweeping optical scanner and the optical focusing system in order to produce an initial horizontal cutting plane, then
the control unit drives the femtosecond laser source, the shaping system, the sweeping optical scanner and the optical focusing system in order to produce at least one vertical cutting plane located above, along the optical axis, the initial horizontal cutting plane, then
the control unit drives the femtosecond laser source, the shaping system, the sweeping optical scanner and the optical focusing system in order to produce a final horizontal cutting plane above, along the optical axis, said and at least one vertical cutting plane.

7. The cutting apparatus according to claim 6, wherein in order to produce a horizontal cutting plane, the control unit:
applies a multipoint phase mask to the shaping system in order to produce a single multipoint modulated laser beam, wherein the multipoint phase mask is calculated to distribute the energy of the multipoint modulated laser beam into at least two impact points in the focal plane of the cutting apparatus,
controls the movement of the focusing system in order to make the focal plane of the cutting apparatus coincide with the desired depth for the horizontal cutting plane,
activates the femtosecond laser source, and
drives the sweeping optical scanner in order to move the impact points of the single multipoint modulated laser beam along a movement path.

8. The cutting apparatus according to claim 6, wherein in order to produce a vertical cutting plane, the control unit:
applies a linear phase mask to the shaping system in order to produce a Bessel modulated laser beam;
controls the movement of the focusing system in order to position the focal plane of the cutting apparatus above or below the desired depth for the vertical cutting plane;
activates the femtosecond laser source; and
drives the sweeping optical scanner in order to move the impact point of the Bessel modulated laser beam along a movement path.
